(19) United States
(12) Patent Application Publication
(10) Pub. No.: US 2006/0036107 A1
(43) Pub. Date: Feb. 16, 2006

(54) Method for producing soy bean oil diols

(76) Inventors: David M. Casper, Normal, IL (US); Trevor Newbold, Bentonville, AR (US)

Correspondence Address: MCKELLAR IP LAW, PLLC, 784 SOUTH POSEYVILLE ROAD, MIDLAND, MI, 48640, US

(21) Appl. No.: 11/195,563

(22) Filed: Aug. 2, 2005

Related U.S. Application Data
(60) Provisional application No. 60/601,315, filed on Aug. 13, 2004.

(51) Int. Cl. C07C 51/16

(52) U.S. Cl. 554/138

(57) ABSTRACT

Combining an aprotic solvent with soybean oil, acid, and hydrogen peroxide at a temperature and for a time sufficient to produce a soybean oil diol.

This application claims priority from U.S. Provisional application 60/601,315 filed on Aug. 13, 2004.

### BACKGROUND

The instant invention is in the field of methods for producing soybean diols.

Soybean oil is primarily a mixture of triglycerides of saturated, unsaturated and polyunsaturated fatty acids. The double bonds of soybean oil are reactive and can be converted to diols by various known methods. Ordinarily, it is desired to convert at least two double bonds of a triglyceride of soybean oil to the diol form so that the resulting soybean diol can be reacted with, for example, toluene diisocyanate to form a linear polyurethane polymer. When three or more double bonds of a triglyceride of soybean oil are converted to diols, then such soybean diol can be reacted with, for example, toluene diisocyanate to form a cross linked polyurethane polymer.

It would be an advance in the area of producing soybean diols if a method were developed that was less cumbersome than the prior art methods.

### SUMMARY OF THE INVENTION

The instant invention provides a facile method for producing soybean diols from soybean diol. The method of the instant invention comprises the step of combining an aprotic solvent with soybean oil, acid, and hydrogen peroxide at a temperature and for a time sufficient to produce the soybean oil diol. In another embodiment of the instant invention, the method comprises the steps of: (a) combining soy bean oil, an aprotic solvent, an acid and hydrogen peroxide to form a reactive mixture, the soybean oil having an equivalent weight equal to the average molecular weight of the soybean oil divided by the average double bond content of the soybean oil, the ration of the number of equivalents of acid to the number of equivalents of soy bean oil being in the range of from one half to five, the ratio of the number of equivalents of hydrogen peroxide to the number of equivalents of soy bean oil being in the range of from two to twenty; and (b) heating the reactive mixture to a temperature and for a time sufficient to produce the soy bean oil diol.

### DETAILED DESCRIPTION OF THE INVENTION

The method of the instant invention comprises the step of combining an aprotic solvent with soybean oil, acid, and hydrogen peroxide at a temperature and for a time sufficient to produce the soybean oil diol. Preferable, the method of the instant invention comprises the steps of: (a) combining soy bean oil, an aprotic solvent, an acid and hydrogen peroxide to form a reactive mixture, the soybean oil having an equivalent weight equal to the average molecular weight of the soybean oil divided by the average double bond content of the soybean oil, the ratio of the number of equivalents of acid to the number of equivalents of soy bean oil being in the range of from one half to five, the ration of the number of equivalents of hydrogen peroxide to the number of equivalents of soy bean oil being in the range of from two to twenty; and (b) heating the reactive mixture to a temperature and for a time sufficient to produce the soybean oil diol.

### COMPARATIVE EXAMPLE 1

One hundred grams of soybean oil having an average of 4.6 double bonds per triglyceride is mixed with 200 milliliters of dioxane. Ten equivalents of hydrogen peroxide per equivalent of soybean oil are added (from 50 weight percent hydrogen peroxide) and the mixture is refluxed for one hour. Analysis of the resulting produce by infra red spectroscopy indicates little soybean diol formation.

### EXAMPLE 1

One hundred grams of soy bean oil having an average of 4.6 double bonds per triglyceride is mixed with 200 milliliters of dioxane. Ten equivalents of hydrogen peroxide per equivalent of soybean oil are added (from 50 weight percent hydrogen peroxide) along with one equivalent of soybean oil and the mixture is refluxed for one hour. Analysis of the resulting product by infra red spectroscopy indicates significant soybean diol formation. Analysis of the resulting product by AOAC official method Cd 13-60 indicates that the hydroxyl value of the soybean diol is 215.

### EXAMPLE 2

One hundred grams of soy bean oil having an average of 4.6 double bonds per triglyceride is mixed with 200 milliliters of dioxane. Ten equivalents of hydrogen peroxide per equivalent of soybean oil are added (from 50 weight percent hydrogen peroxide) along with two equivalents of soybean oil and the mixture is refluxed for one hour. Analysis of the resulting product by infra red spectroscopy indicates significant soybean diol formation. Analysis of the resulting product by AOAC official method Cd 13-60 indicates that the hydroxyl value of the soybean diol is 224.

### EXAMPLE 3

One hundred grams of soy bean oil having an average of 4.6 double bonds per triglyceride is mixed with 200 milliliters of dioxane. Ten equivalents of hydrogen peroxide per equivalent of soybean oil are added (from 50 weight percent hydrogen peroxide) along with three equivalents of acid (from concentrated sulfuric acid) per equivalent of soy bean oil and the mixture is refluxed for one hour. Analysis of the resulting product by infra red spectroscopy indicates significant soybean diol formation. Analysis of the resulting product by AOAC official method Cd 13-60 indicates that the hydroxyl value of the soybean diol is 203.

### EXAMPLE 4

One hundred grams of soy bean oil having an average of 4.6 double bonds per triglyceride is mixed with 200 milliliters of dioxane. Five equivalents of hydrogen peroxide per equivalent of soybean oil are added (from 50 weight percent hydrogen peroxide) along with one equivalent of acid (from concentrated sulfuric acid) per equivalent of soybean oil and the mixture is refluxed for one hour. Analysis of the resulting product by infra red spectroscopy indicates significant soybean diol formation. Analysis of the resulting product by AOAC official method Cd 13-60 indicates that the hydroxyl value of the soybean diol is 190.

### EXAMPLE 5

One hundred grams of soy bean oil having an average of 4.6 double bonds per triglyceride is mixed with 200 milliliters of dioxane. Seven equivalents of hydrogen peroxide per equivalent of soybean oil are added (from 50 weight percent hydrogen peroxide) along with one equivalent of acid (from concentrated sulfuric acid) per equivalent of soybean oil and the mixture is refluxed for one hour. Analysis of the resulting product by infra red spectroscopy indicates significant soybean diol formation. Analysis of the resulting product by AOAC official method Cd 13-60 indicates that the hydroxyl value of the soybean diol is 196.

### EXAMPLE 6

One hundred grams of soy bean oil having an average of 4.6 double bonds per triglyceride is mixed with 200 milliliters of dioxane. Seven equivalents of hydrogen peroxide per equivalent of soybean oil are added (from 50 weight percent hydrogen peroxide) along with one equivalent of acid (from concentrated sulfuric acid) per equivalent of soybean oil and the mixture is refluxed for one hour. Analysis of the resulting product by infra red spectroscopy indicates significant soybean diol formation. Analysis of the resulting product by AOAC official method Cd 13-60 indicates that the hydroxyl value of the soybean diol is 156.

### EXAMPLE 7

One hundred grams of soy bean oil having an average of 4.6 double bonds per triglyceride is mixed with 200 milliliters of dioxane. Nine equivalents of hydrogen peroxide per equivalent of soybean oil are added (from 50 weight percent hydrogen peroxide) along with one equivalent of acid (from concentrated sulfuric acid) per equivalent of soybean oil and the mixture is refluxed for one hour. Analysis of the resulting product by infra red spectroscopy indicates significant soybean diol formation. Analysis of the resulting product by AOAC official method Cd 13-60 indicates that the hydroxyl value of the soybean diol is 201.

Although dioxane is used as the solvent in the above examples, and although dioxane is the preferred aprotic solvent in the method of the instant invention, any suitable aprotic solvent can be used in the method of the instant invention. Although sulfuric acid is used as the source of acid in the above examples, and although sulfuric acid is the preferred source of acid in the method of the instant invention, any suitable source of acid can be used in the method of the instant invention. Although 50 weight percent hydrogen peroxide is used as the source of hydrogen peroxide in the above examples and although 50 weight percent hydrogen peroxide is preferred as the source of hydrogen peroxide in the instant invention it should be understood that, of course, other concentrations of hydrogen peroxide or other sources of hydrogen peroxide can be used in the instant invention.

What is claimed is:

1. A method for producing soybean oil diols from soy bean oil, comprising the step of: combining an aprotic solvent with soybean oil, acid, and hydrogen peroxide at a temperature and for a time sufficient to produce the soybean oil diol.
